(19) United States
(12) Patent Application Publication
(10) Pub. No.: US 2024/0140899 A1
(43) Pub. Date: May 2, 2024

(54) SMB SEPARATOR FOR ORGANIC ACID PURIFICATION USING A STRONG ACID CATION RESIN

(71) Applicant: AMALGAMATED RESEARCH LLC, Twin Falls, ID (US)

(72) Inventors: Lily Crane, Twin Falls, ID (US); Peter V. Ferrero, Twin Falls, ID (US)

(73) Assignee: AMALGAMATED RESEARCH LLC, Twin Falls, ID (US)

(21) Appl. No.: 18/499,697

(22) Filed: Nov. 1, 2023

Related U.S. Application Data
(60) Provisional application No. 63/421,908, filed on Nov. 2, 2022.

(51) Int. Cl. C07C 51/47; B01D 15/18; B01D 15/36; B01J 39/05; B01J 39/18; B01J 47/10

(52) U.S. Cl. CPC C07C 51/47; B01D 15/1828; B01D 15/1842; B01D 15/362; B01J 39/05; B01J 39/18; B01J 47/10

(57) ABSTRACT

Systems and methods for enriching an organic acid from a fermentation broth containing residual color, salts, and carbohydrates using simulated moving bed (“SMB”) chromatography to produce a high purity organic acid extract is disclosed. One embodiment of the method purifies citric acid from a fermentation broth into two product streams: a first product stream rich in salt, color, and carbohydrate impurities, and a second product stream comprising an extract enriched in organic acid.

### CROSS-REFERENCE TO RELATED APPLICATIONS

This application, under 35 U.S.C. § 119, claims the benefit of U.S. Provisional Patent Application Ser. No. 63/421,908 filed on Nov. 2, 2022, and entitled “SMB Separator For Organic Acid Purification Using A Strong Acid Cation Resin,” the contents of which are hereby incorporated by reference herein.

### FIELD OF THE DISCLOSURE

This disclosure relates generally to simulated moving bed (SMB) separation and purification systems and methods. In particular, this disclosure relates to systems and methods for an SMB separator for organic acid purification using a strong acid cation resin.

### BACKGROUND

Organic acids such as lactic acid, malic acid, fumaric acid, and citric acid are widely used in the food industry as flavorings and acidulants. Industrially the upstream process for producing many commercially available organic acids typically involves the fermentation of either cane or beat sugar molasses or corn syrups. For example, molasses is commonly used due to the integrated sugars, amino acids, and salts aiding in the growth of the bacteria or yeast to produce the desired organic acid. After fermentation is complete, the fermentation broth containing the organic acid product must undergo several purification steps to reduce the residual sugar, ash, and color content of the fermentation broth before crystallizing the organic acid. It is common to utilize a two-step precipitation process to remove most of the color and monovalent salts from the fermentation broth. First, calcium carbonate is added to the fermentation broth to precipitate the insoluble calcium salt of the organic acid product as detailed in, for example, U.S. Pat. Nos. 2,389,766 and 3,798,266 for which the contents of both are hereby incorporated by reference. The precipitated organic acid is thoroughly washed to remove the residual broth containing the color and monovalent salt impurities. Following this, sulfuric acid is used to redissolve the organic acid and precipitate the calcium as calcium sulfate or gypsum. The gypsum is then filtered from the dissolved organic acid solution which is then fed to an evaporator for concentration before crystallization. This process typically produces approximately one ton of gypsum waste per ton of organic acid produced which must be valorized which is problematic from an environmental perspective. A variation of this method is taught in U.S. Pat. No. 9,376,364, the contents of which is hereby incorporated by reference, and uses magnesium carbonate for the precipitation process which is then recycled through calcination which is energy intensive.

A technology that has been historically investigated into for the purification of these organic acids is ion exchange, as it would eliminate the gypsum byproduct waste of the gypsum purification process. These methods use various types of anion exchange resin to bind the deprotonated organic acid followed by elution with salt, caustic, or acid as taught in EP2592952B1, U.S. Pat. Nos. 6,641,734, 5,382,681, 6,087,139, and 2,664,441 for which the contents of each are hereby incorporated by reference. This facilitates the removal of the residual sugar, ash, and color impurities in the fermentation broth. However, a severe disadvantage of using anion exchange for organic acid purification is the high amount of chemical required during the elution step of the ion exchange cycle. Oftentimes, the amount of chemical required can exceed three equivalents per equivalent of organic acid recover which represents a significant cost in terms of both purchasing and disposing of salt, caustic, or acid regenerants. It is thus desirable to identify alternative resin-based methods to purify organics acids while minimizing the amount of chemicals consumed and waste by-products produced over the course of the purification process. Attempts to minimize the mineral acid usage through SMB chromatography using weak base anion exchange resins have been detailed in U.S. Ser. No. 10/279,282B2 and U.S. Pat. No. 9,776,945B2, for which the contents of each are incorporated herein by reference. Other drawbacks and inefficiencies also exist with current systems and methods.

Strong acid cation (SAC) exchange resins have been extensively used to purify neutral sugars such as sucrose, glucose, and fructose in the sweetener industries using SMB chromatography. The eluent for regenerating the SAC resin is simply condensate water produced elsewhere in the plant. This minimizes the amount of chemical consumed and disposed of compared to an ion exchange process. It was postulated that under certain conditions, organic acids could be purified from fermentation broths using a SAC resin instead of anion exchange as is typically practiced. However, SAC resins are typically used to purify positively-charged compounds from impurities. The use of a cation exchange resin to purify organic acids which tend to dissociate into negatively-charged compounds is not known to be used for purification by those skilled in the art. Other drawbacks and inefficiencies also exist with current systems and methods.

### SUMMARY

Accordingly, presently disclosed systems and methods are directed to addressing the above, and other, drawbacks and inefficiencies of existing systems and methods. It was postulated that under certain conditions, organic acids could be purified from fermentation broths using a SAC resin instead of anion exchange as is typically practiced. It was also postulated that an organic acid may be purified using a cation exchange resin provided that the pH of the fermentation broth was sufficiently low so that the total amount of organic acid in the broth was protonated and thus of neutral charge. This would allow the use of SMB chromatography to purify the organic acid in a similar manner to the purification of sucrose from sugar beet molasses. This approach retains the advantage of only requiring sulfuric acid to lower the pH of the fermentation broth to ensure the organic acid to be purified is fully protonated in the free acid form.

An additional advantage from this process is that unlike anion exchange resins, cation exchange resins are resistant to organic foulants such as dextran and polysaccharides often found in fermentation broths. Furthermore, SAC resins are available in smaller resin particle sizes compared to anion exchange resins which further reduce the resin and eluent requirements for the SMB process utilizing a SAC resin.

Disclosed embodiments include processes for purifying an organic acid including separating an organic acid from a fermentation broth (FB) by adding an acid to the FB to form protonated organic acid, creating a solution of dissolved solids from the protonated organic acid, processing the solution of dissolved solids by using it as feedstock in a simulated moving bed (SMB) chromatography system that uses a dilute acid as an eluent and a strong acid cation (SAC) exchange resin, and wherein each step of the SMB chromatography system is divided into two sub-periods wherein a first sub-period encompasses a span of time where the feedstock and the eluent are injected into distinct columns within a recirculation loop and, concurrently, extract and raffinate fractions are also withdrawn from the SMB chromatography system at defined points and during a second sub-period an internal solids profile is recirculated within the SMB chromatography system without any additional material added or removed.

In some embodiments the FB comprises salt, color waste, fermentable sugars. In some embodiments the organic acid is selected from the group including, but not limited to, citric acid, malic acid, fumaric acid, acetic acid, tartaric acid, glycolic acid, glucaric acid, lactic acid, xylonic acid, gluconic acid, galactaric acid, succinic acid, maleic acid, itaconic acid, malonic acid, terephthalic acid, phthalic acid, glutaric acid, adipic acid, 3-hydroxyproponic acid, formic acid, and oxalic acid.

In some embodiments the process of purifying an organic acid includes adding an acid to the FB to lower the pH of the FB to be substantially between 1.0-2.0. In some embodiments, the step of adding an acid to the FB includes adding 93% concentrated sulfuric acid.

In some embodiments the eluent used in the SMB chromatography system comprises acidified water where the acid used is a mineral acid. In some embodiments the acidified water includes, but is not limited to, sulfuric acid, hydrochloric acid, nitric acid, or phosphoric acid. In some embodiments the process of purifying an organic acid includes adjusting the pH of the acidified water to be substantially between 1.0-2.0.

Also disclosed are systems for purifying an organic acid including a simulated moving bed (SMB) chromatography system for processing a solution of dissolved solids by using it as feedstock and that uses a dilute acid as an eluent, a strong acid cation (SAC) exchange resin, and wherein each step of the SMB chromatography system is divided into two sub-periods wherein a first sub-period encompasses a span of time where the feedstock and the eluent are injected into distinct columns within a recirculation loop and, concurrently, extract and raffinate fractions are also withdrawn from the SMB chromatography system at defined points and during a second sub-period an internal solids profile is recirculated within the SMB chromatography system without any additional material added or removed.

In some embodiments, the solution of dissolved solids is formed by separating an organic acid from a fermentation broth (FB) by adding a mineral acid to the FB to form protonated organic acid. In some embodiments the mineral acid is selected from the group including, but not limited to, sulfuric acid, hydrochloric acid, nitric acid, or phosphoric acid.

In some embodiments the FB comprises salt, color waste, fermentable sugars. In some embodiments the organic acid is selected from the group including, but not limited to, citric acid, malic acid, fumaric acid, acetic acid, tartaric acid, glycolic acid, glucaric acid, lactic acid, xylonic acid, gluconic acid, galactaric acid, succinic acid, maleic acid, itaconic acid, malonic acid, terephthalic acid, phthalic acid, glutaric acid, adipic acid, 3-hydroxyproponic acid, formic acid, and oxalic acid.

In some embodiments system for purifying an organic acid included an acid added to the FB to lower the pH of the FB to be substantially between 1.0-2.0. In some embodiments the acid added to the FB comprises 93% concentrated sulfuric acid. In some embodiments the system for purifying an organic acid includes adjusting the pH of the mineral acid to be substantially between 1.0-2.0. Other advantages, efficiencies, and features of disclosed systems and processes also exist.

### BRIEF DESCRIPTION OF THE DRAWINGS

FIG. 1 illustrates the disclosed exemplary embodiments' effectiveness of using a SAC resin in an SMB system to purify organic acids from sucrose, ash, and color.

FIG. 2 depicts an SMB system to produce purified organic acid from a fermentation broth according to embodiments of the disclosure.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the spirit and scope of the invention as defined by the appended claims.

### DETAILED DESCRIPTION

The following examples serve to explain the embodiments of the disclosure in more detail. These examples are not to be construed as being exhaustive or exclusive as to the scope of this disclosure and other embodiments will be apparent to those of ordinary skill in the art having the benefit of this disclosure.

In one exemplary embodiment, a fermentation broth containing approximately 30,000 ppm salt, 275.0 absorbance units of color, 1-2 w/w % sucrose and 7-8 w/w % organic acid was adjusted with 93% concentrated sulfuric acid to lower the pH to between 1.0-2.0 to fully protonate the target organic acid to be purified.

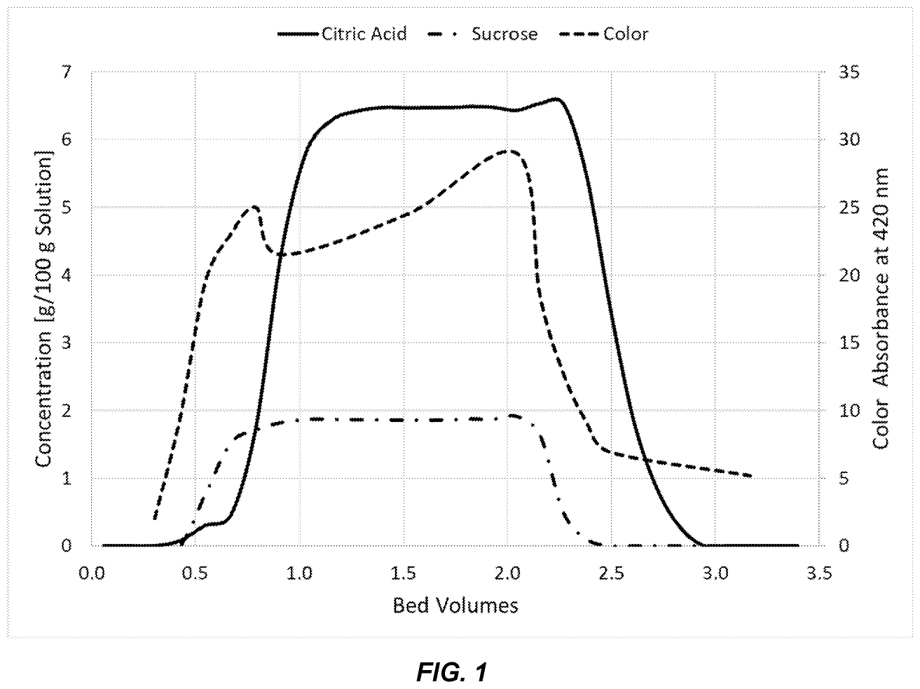

To ensure that the organic acid remained in the protonated form throughout the entire batch test, the deionized (DI) water used as eluent was also pH adjusted to 1.0-2.0 pH with 93% concentrated sulfuric acid. Otherwise, the more neutral pH of DI water would raise the pH of the fermentation broth causing the citric acid to deprotonate. To stop this deprotonation from occurring it was also important that the starting fluid in the column be the pH adjusted eluent, and that the resin be soaked in the eluent prior to beginning a run of the process. The results from one of the exemplary runs are shown FIG. 1.

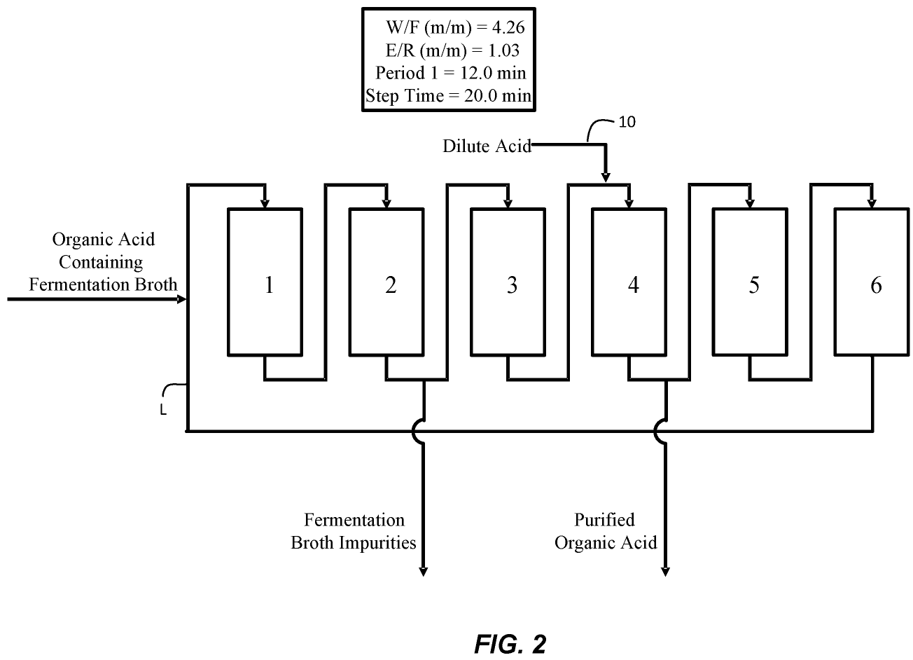

Following confirmation of separation between the organic acid and salt, color, and carbohydrate impurities, a SMB chromatography system was used to purify the organic acid from the various other impurities on a continuous basis. A solution containing 30% total dissolved solids of a citric acid containing material was processed using the SMB system as configured in FIG. 2 in which items 1-6 indicate individual columns of the SMB. A solution of diluted sulfuric acid at pH 1.5 was used as the eluent for the SMB system as indicated at 10 on FIG. 2. In the context of operating an SMB unit, “step” is herein defined as the subset of operation wherein the feedstock is introduced (e.g., injected) into a single column (e.g., 1-6) upon which at the end of the “step” the configuration of valves advances such that material is introduced into the next downstream column within the recirculation loop L. The SMB separation of the SMB system was operated such that each step was divided into two sub-periods as described in U.S. Pat. No. 5,102,553, the disclosure of which is incorporated by reference herein. The first sub-period encompassed a span of time where the feedstock and eluent were injected into distinct columns within the recirculation loop (for example, as shown at column 1 and 4, respectively). Concurrently, extract and raffinate fractions were also withdrawn from the SMB separator at defined points (for example, as shown between columns 2 and 3 and columns 4 and 5, respectively). During the second sub-period, the internal solids profile within the SMB separator recirculated through the SMB system without any additional material added or removed from the SMB separator. The SMB separator was filled with Mitsubishi UBK-522M strong acid cation resin in the potassium form. The SMB separator was loaded at approximately 45.9 lbs. dissolved solids per cubic foot resin per day (approximately 736 kilograms dissolved solids per cubic meter resin per day). The total resin volume for this experiment was 0.65 cubic feet (18.5 liters) distributed amongst 6 columns. The product and by-product stream produced by the SMB chromatography system and the associated operating parameters of the system are listed in Table 1 below.

TABLE 1
SMB Operating Parameters

| | | |
|---|---|---|
| Resin Solids | (lb DS/ft [symbol] | 45.9 |
| Loading | resin/day) | |
| Water-to-Feed | (mass ratio) | 4.26 |
| Ratio | | |
| Extract-to- | (mass ratio) | 1.03 |
| Raff [symbol] Ratio | | |
| Period 1 Time | ( [symbol] ) | 12 |
| Step Time | ( [symbol] ) | 20 |

SMB Performance

| | | Citric Acid | Sucrose | | Color | Citric | | |
|---|---|---|---|---|---|---|---|---|
| | Dissolved | Purity | Purity | Total | by | Acid | Impurity | Color |
| | Solids | (g/100 g | (g/100 g | Salt | Absorb- | Recovery | Removal | Removal |
| Stream | (%) | DS) | DS) | (ppm) | ance | (%) | (%) | (%) |
| Feed | 39.80 | [symbol] 4 | 8.83 | 29788 | 275.0 | 93.9 | [symbol] 2.9 | [symbol] 7.0 |
| Extract | [symbol] | 8 [symbol] | 0.2 [symbol] | 3100 | 23.4 | | | |
| Raff [symbol] | [symbol] | 7.16 | 18.62 | [symbol] 357 | [symbol] 2.5 | | | |

[symbol]

indicates data missing or illegible when filed

Although various embodiments have been shown and described, the present disclosure is not so limited and will be understood to include all such modifications and variations would be apparent to one skilled in the art.

What is claimed is:

1. A process for purifying an organic acid comprising:
separating an organic acid from a fermentation broth (FB) by adding an acid to the FB to form protonated organic acid;
creating a solution of dissolved solids from the protonated organic acid;
processing the solution of dissolved solids by using it as feedstock in a simulated moving bed (SMB) chromatography system that uses a dilute acid as an eluent and a strong acid cation (SAC) exchange resin; and
wherein each step of the SMB chromatography system is divided into two sub-periods wherein a first sub-period encompasses a span of time where the feedstock and the eluent are injected into distinct columns within a recirculation loop and, concurrently, extract and raffinate fractions are also withdrawn from the SMB chromatography system at defined points and during a second sub-period an internal solids profile is recirculated within the SMB chromatography system without any additional material added or removed.

2. The process of purifying an organic acid of claim 1 wherein the FB comprises salt, color waste, fermentable sugars.

3. The process of purifying an organic acid of claim 1 wherein the organic acid is selected from the group consisting of: citric acid, malic acid, fumaric acid, acetic acid, tartaric acid, glycolic acid, glucaric acid, lactic acid, xylonic acid, gluconic acid, galactaric acid, succinic acid, maleic acid, itaconic acid, malonic acid, terephthalic acid, phthalic acid, glutaric acid, adipic acid, 3-hydroxyproponic acid, formic acid, and oxalic acid.

4. The process of purifying an organic acid of claim 1 further comprising:
adding an acid to the FB to lower the pH of the FB to be substantially between 1.0-2.0.

5. The process of purifying an organic acid of claim 4 wherein adding an acid to the FB comprises adding 93% concentrated sulfuric acid.

6. The process of purifying an organic acid of claim 1 wherein the eluent used in the SMB chromatography system comprises acidified water where the acid used is a mineral acid.

7. The process of purifying an organic acid of claim 6 wherein the acidified water comprises one of the group consisting of sulfuric acid, hydrochloric acid, nitric acid, or phosphoric acid.

8. The process of purifying an organic acid of claim 7 further comprising adjusting the pH of the acidified water to be substantially between 1.0-2.0.

9. A system for purifying an organic acid comprising:
a simulated moving bed (SMB) chromatography system for processing a solution of dissolved solids by using it as feedstock and that uses a dilute acid as an eluent, a strong acid cation (SAC) exchange resin; and
wherein each step of the SMB chromatography system is divided into two sub-periods wherein a first sub-period encompasses a span of time where the feedstock and the eluent are injected into distinct columns within a recirculation loop and, concurrently, extract and raffinate fractions are also withdrawn from the SMB chromatography system at defined points and during a second sub-period an internal solids profile is recirculated within the SMB chromatography system without any additional material added or removed.

10. The system for purifying an organic acid of claim 9 wherein the solution of dissolved solids is formed by separating an organic acid from a fermentation broth (FB) by adding a mineral acid to the FB to form protonated organic acid.

11. The system for purifying an organic acid of claim 10 wherein the mineral acid is selected from the group consisting of: sulfuric acid, hydrochloric acid, nitric acid, or phosphoric acid.

12. The system for purifying an organic acid of claim 9 wherein the FB comprises salt, color waste, fermentable sugars.

13. The system for purifying an organic acid of claim 9 wherein the organic acid is selected from the group consisting of: citric acid, malic acid, fumaric acid, acetic acid, tartaric acid, glycolic acid, glucaric acid, lactic acid, xylonic acid, gluconic acid, galactaric acid, succinic acid, maleic acid, itaconic acid, malonic acid, terephthalic acid, phthalic acid, glutaric acid, adipic acid, 3-hydroxyproponic acid, formic acid, and oxalic acid.

14. The system for purifying an organic acid of claim 9 further comprising:
an acid added to the FB to lower the pH of the FB to be substantially between 1.0-2.0.

15. The system for purifying an organic acid of claim 14 wherein the acid added to the FB comprises 93% concentrated sulfuric acid.

16. The system for purifying an organic acid of claim 10 further comprising adjusting the pH of the mineral acid to be substantially between 1.0-2.0.
